(19) United States
(12) Patent Application Publication
(10) Pub. No.: US 2018/0021347 A1
(43) Pub. Date: Jan. 25, 2018

(54) COMPOSITION FOR THE TREATMENT OF HEPATIC VENO-OCCLUSIVE DISEASE

(71) Applicant: SCIPHARM SÀRL, Luxemburg (LU)

(72) Inventors: Michael Freissmuth, Wien (AT); Eva-Maria Zebedin-Brandl, Wien (AT); Zahra Kazemi, Wien (AT); Christoph Österreicher, Wien (AT); Ursula Lemberger, Wien (AT); Madeleine Themanns, Wien (AT)

(21) Appl. No.: 15/547,060

(22) PCT Filed: Jan. 27, 2016
(86) PCT No.: PCT/EP2016/051675

§ 371 Date: Jul. 27, 2017

(30) Foreign Application Priority Data
Jan. 27, 2015 (EP) 15152665.4

(51) Int. Cl. A61K 31/5575; A61K 9/00

(52) U.S. Cl. CPC A61K 31/5575; A61K 9/0019; A61K 9/0073

(57) ABSTRACT

The present invention provides a composition comprising a prostacyclin analogue, derivative or a pharmaceutically acceptable salt thereof, for use in preventing or treating the sinusoidal obstruction syndrome and/or hepatic veno-occlusive disease (VOD) as well as treatment regimens therefor.

The present invention provides for compositions comprising a prostacyclin or a prostacyclin analogue or a pharmaceutically acceptable salt thereof for use in preventing or treating sinusoidal obstruction syndrome and/or hepatic veno-occlusive disease (VOD).

Hepatic complications of hematopoietic cell transplantation (HCT) have been a common cause of morbidity and mortality. Hepatic veno-occlusive disease (VOD) is a life-threatening complication of the intensive chemotherapy regimens used in allogeneic/autologous stem cell transplantation (SCT). The name sinusoidal obstruction syndrome is often used if VOD happens as a result of chemotherapy and/or bone marrow transplantation.

The onset of VOD is typically by day +35 after SCT. VOD is part of a spectrum of organ injury syndromes that occur after high-dose chemotherapy, with or without irradiation and SCT, including idiopathic pneumonitis, diffuse alveolar hemorrhage, thrombotic microangiopathy and capillary leak syndrome (Wadleigh M. et al., 2003, Curr.Opin.Hematol., 10, 451-462).

Severe VOD complicated by multisystem organ failure (MOF) remains almost uniformly fatal. The clinical syndrome is characterized by painful hepatomegaly, jaundice, ascites and fluid retention (unexplained weight gain), which occur in 10-60% of patients undergoing high-dose chemotherapy and SCT. The condition ranges in severity from a mild reversible disease to a severe disease culminating in multiorgan failure (MOF) and death (Richardson P. et al., 2002, Blood, 4337-4342; Jones et al, 1987, Transplant, 44, 778-783).

The causes of VOD are still unclear, but a combination of pre-transplant risk factors and transplant-related conditions are believed to trigger a primarily hepatic sinusoidal injury. This can quickly extend to a hepatocytic and panvasculitic disease, which is followed by multiorgan failure that is associated with substantial mortality. The initiating pathophysiological events have prompted the suggestion that this form of liver disease be renamed sinusoidal obstruction syndrome (SOS).

The risk of veno-occlusive disease in the pediatric population is not limited to a well-defined group of high-risk patients who have undergone transplantation. The disease frequently occurs outside this group. For example, patients treated for solid tumors (eg., Wilms tumor, neuroblastomas, and rhabdomyosarcomas) are at a high risk for developing VOD.

Single nucleotide polymorphisms of the donor may also be a factor in the onset of VOD in children receiving an allogeneic transplant.

The pathophysiology of VOD remains obscure. Injury to the hepatic venules is believed to represent the first histological change in VOD. VOD is thought to originate from damage to sinusoidal endothelial cells and hepatocytes in zone 3 of the liver acinus surrounding the central veins. Early changes include deposition of fibrinogen, factor VIII, and fibrin within venular walls and sinusoids. Subendothelial edema, collagen deposition, sclerosis, and fibrosis of the abluminal venular area follow, with stellate cell proliferation and collagenization contributing to matrix deposition. As the process of venular microthrombosis, fibrin deposition, ischemia, and fibrogenesis advances, widespread zonal disruption leads to portal hypertension, hepatorenal syndrome, MOF, and death (Shulman et al, 1987, Am. J. Pathol., 127, 549-558; 1994, Hepatology, 19, 1171-1181). During this period, a procoagulant state is present with low plasma levels of anti-thrombin III and protein C, consumption of Factor VII and increased levels of plasminogen activator inhibitor 1 (PAI-1) (Salat et al, 1997, Blood, 89, 2184-2188). Increased levels of von Willebrand factor (vWF) multimers, an increase in platelet adhesion and refractoriness to platelet transfusions are indicative of ongoing endothelial cell injury (Palomo M et al, Biol Blood Marrow Transplant 16(7):985-93, 2010).

Hepatic sinusoidal obstruction syndrome (HSOS) is a new name given to VOD of the liver, since VOD can develop without venular involvement. In experimental studies of monocrotaline-induced VOD rats the obstruction has been shown to originate from sinusoidal endothelial cells (SECs) rather than from hepatocytes. Early signs of endothelial cell damage were evident already 24 h after a single gavage of monocrotaline. The disease eventually presented as an obliterative venulitis of the terminal hepatic venules (DeLeve L D, AmJPhysiolGastrointestLiverPhysiol 284:G1045
G1052, 2003).

Interestingly, recently it has been recognized that this pathological condition may not be restricted to the liver. The lung has been suggested as a potential “venue” for VOD: in rare cases, VOD might here precede the development of pulmonary artery hypertension (PAH), in particular following mitomycin-induced endothelial damage (Masters K et al, BMJ Case Rep, bcr2012007752, 2013). Similar to VOD that occurs after SCT, VOD arising after exposure to gemtuzumab ozogamizin (Mylotarg), an anti-CD33 immunotoxin is also characterized by marked sinusoidal obstruction and intense fibrosis (Wadleigh, 2003). Cytotoxic drugs used in SCT, like busulphan or the metabolites of cyclophosphamide, for example acrolein or 4-hydroxy cyclophosphamide, are also associated with an increased risk of VOD.

Early identification of high-risk patients with severe disease is of importance because of the high mortality rates associated with severe VOD.

The severity of VOD is divided into the following three categories:

Mild Disease
No adverse effects from veno-occlusive disease
No treatment necessary
Self-limiting

Moderate Disease
No adverse effects from veno-occlusive disease
Requires treatment (pain medication, diuretics, other supportive care)

Severe Disease
Unresolved signs and symptoms of veno-occlusive disease 100 days after stem cell transplantation
Death due to complications directly attributable to veno-occlusive disease.

Severe veno-occlusive disease was more precisely defined based on the presence of multiorgan failure in addition to veno-occlusive disease. Multiorgan failure is characterized by oxygen requirement (with an oxygen saturation of <90% on room air, ventilator dependence, or both), renal dysfunction (defined as doubling of baseline creatinine levels, dialysis dependence, or both), and/or encephalopathy (Harper J.L., emedicine.medscape.com, Veno-occlusive hepatic disease, 2012).

Markers of endothelial injury in VOD include plasma thrombomodulin, P-selectin and plasminogen activator inhibitor (PAI), tissue factor pathway inhibitor, soluble tissue factor, thrombomodulin and P- and E-selectin and activated hepatic stellate perisinusoidal cells. Tumor necrosis factor alpha, interleukin 6, IL-8 and IL-113 may contribute to initial endothelial injury. Elevation of transforming growth factor beta, collagen propeptide, hyaluronic acid and immunopeptide of type 3 procollagen (PIIINP) have been observed in VOD (Wadleigh, 2003).

The clinical diagnosis of VOD is based on weight gain, painful hepatomegaly and jaundice. Transvenous liver biopsy and wedged hepatic venous pressure gradient measurement (WHVPG) remain gold standards of the pathologic diagnosis of VOD.

Despite therapeutic interventions, including the use of antithrombotic and thrombolytic agents such as prostaglandin E1 and tissue-plasminogen activator (t-PA) with or without concurrent heparin, little success has been achieved in the treatment of severe VOD (Richardson P. et al., 2001, Acta Haematol., 106, 57-68; Baglin T P. et al., 1990, Bone Marrow Transplant, 5, 439-441). In aggregate, despite multiple interventions and intensive treatment, day+100 mortality for severe VOD has remained in excess of 90% (Richardson P. et al., 2001, 2002, Carreras E. et al., 1998, Blood, 92, 3599-3604).

Senzolo M. et al. describe the clinical treatment regimens of VOD. It is reported therein that prostaglandin E2 used in combination with heparin showed lower incidence of VOD, whereas PGE1 alone failed to show any advantage (2007, World J. Gastroenterol., 13(29), 3918-3924).

WO2004019952A1 describes the use of prostacyclin analogies for improving venous flow.

Witt W. et al. describe the use of Iloprost for the treatment of patients with thrombotic diseases (1985, “Antithrombotic profile of Iloprost in experimental models of arterial and venous thrombosis, Springer Verlag, 81-90).

CA2306567A1 reports a plasminogen activator inhibitor 1 containing a cAMP enhancer, e.g. forskolin or adenylate cyclase.

Alliot C et al., Presse Médicale, 1994, vol. 23, no. 40, p. 1878 disclose the use of the prostacyclin Flolan at a maximum tolerable dosage of 5 to 8 ng/kg/day for the treatment of VOD in a single patient.

Presently there are no treatments available that significantly improve therapy regimens and there is still an unmet need to provide therapy for the treatment of VOD. VOD is the dose-limiting toxicity for several chemotherapeutic drugs and limits patient eligibility. A prophylactic treatment of VOD would have a significant impact on the ability to use high dose chemotherapy. Development of therapies to treat VOD after onset of the disease would also be of value in unexpected cases of chemotherapy-induced liver disease.

Therefore it is the object of the invention to provide compositions for improved therapy of patients suffering from or being at risk of VOD or sinusoidal obstruction syndrome as result of chemotherapy.

### SHORT DESCRIPTION OF THE INVENTION

The objective is specifically solved by the claimed subject matter.

According to the invention, there is provided a composition comprising an active agent, which is selected from the group consisting of prostacyclin, a prostacyclin analogue, derivative or a pharmaceutically acceptable salt thereof, for use in preventing or treating hepatic veno-occlusive disease (VOD) or, specifically, sinusoidal obstruction syndrome.

According to a preferred embodiment the composition comprises a prostacyclin analogue, derivative or a pharmaceutically acceptable salt thereof. Specifically said prostacyclin analogues are selectively stimulating EP₂ and EP₄ receptors optionally in addition to the I prostanoid receptors (IP) but are low affinity agonists at inhibitory Gᵢ-coupled EP₃ receptors.

Specifically, said prostacyclin analogue is selected from the group of Treprostinil, Iloprost, Cicaprost or Beraprost, derivatives or pharmaceutically acceptable salts thereof.

Specifically, said derivative is selected from the group of acid derivatives of Treprostinil, prodrugs of Treprostinil, sustained release forms of Treprostinil, inhaled forms of Treprostinil, oral forms of Treprostinil, polymorphs of Treprostinil or isomers of Treprostinil.

According to a specific aspect, the composition is provided for systemic administration, preferably by intravenous or subcutaneous infusion. A continuous infusion over a prolonged period of time is envisaged, e.g. for at least 1 hour, preferably at least 2 hours, preferably at least 5 hours, more preferably at least 24 hours, preferably at least 7 days, specifically up to 365 days.

According to a further specific aspect, the composition is provided for local administration, preferably for inhalation. Therefore, a suitable inhalator is provided that provides for administration of an effective amount of the drug.

According to a further specific aspect, the composition is provided for oral treatment, e.g. wherein said composition is in an orally available form selected from the group of tablets or capsules.

Specifically the composition is a pharmaceutical composition, preferably comprising one or more pharmaceutically acceptable carriers and/or additives.

The dosage of the active agent in the composition of the invention depends on different parameters, e.g. the patient to which the active compound is administered, specifically the patient's body weight and age, the patient's individual condition, the disease and severity of the disease, and the route and frequency of administration. The active compound may e.g. be administered orally in a dose of 0.1 −1000 mg/kg per day, preferably 5-700 mg/kg, preferably 5-500 mg/kg per day, which can be split up into several, e.g. 1, 2, 3 or more doses. For an inhalative administration the preferred dose of the active compound per inhalation is between 100 pg-1000 mg/kg/day.

According to an alternative embodiment, a dosage container for repeated inhalative administration is provided, wherein said container contains about 20 mg, specifically 19, 18, 17, 16, 15 mg active compound, specifically Treprostinil.

According to a further embodiment, inhalative administration is specifically useful for VOD treatment wherein the lung is the venue for VOD.

Several of such doses can be administered successively and/or several times a day if needed.

Specifically, the composition is a pharmaceutical composition that provides for an effective amount of Treprostinil or its derivative, or a pharmaceutically acceptable salt thereof, which is ranging between about 0.1 ng/kg/min to 100 ng/kg/min, preferably a daily dose from about 1 to 50 ng/kg/min, preferably between about 10 to 45 ng/kg/min, more preferably between about 20 to 40 ng/kg/min.

Preferred compositions provide for an effective amount of inhaled Treprostinil ranging between 10 and 200 μg/kg, specifically between 25 to 150 μg/kg, more specifically between 50 and 100 μg, even more specifically between about 50 to 60 μg/kg, preferably administered by 2 to 6 treatments per day, e.g. 2, 3, 4 or more separate treatment sessions, equally spaced during the day. Typically 5 to 150 μg/kg Treprostinil is administered per treatment session, e.g. 10 to 100 pg/kg, preferably starting with lower doses of 10 to 30 pg/kg followed by higher doses of 40 to 100 pg/kg, preferably about 40 to 70 pg/kg.

Preferably a nebulizer is used which generates a pulsed aerosol cloud of formulation. Each pulse delivers, e.g. 5-10 pg/kg, such as 6 pg/kg of Treprostinil from the mouthpiece.

According to an embodiment of the invention, a metered dose inhaler can be used for administration which may be a device capable of delivering a metered or bolus dose of the prostacyclin analogue or derivative of the invention to the lungs of a VOD patient. It may be for example a pressurized metered dose inhaler (pMDI), i.e. a device which produces aerosol clouds for inhalation from solutions or suspensions of the prostacyclin analogue or derivatives.

The inhalation device may also be a dry powder inhaler (DPI) wherein the prostacyclin analogue or derivative is present as solid formulation.

As an alternative, the metered dose inhaler can be a soft mist inhaler in which the aerosol cloud can be generated by passing a solution through a nozzle or a series of nozzles. Examples of said inhalers are available as Respimat® Inhaler (Boehringer Ingelheim), AERx® Inhaler (Aradigm Corp.), Mystic™ Inhaler (Ventaira Pharmaceuticals, Inc) or Aira™ Inhaler (Chrysalis Technologies Inc.).

Treprostinil can also be preferably administered by infusion, e.g. in an amount which is at least 0.1 ng/kg of body weight/min, preferably ranging between 0.5 ng/kg and 1 mg/kg/min, preferably between 1 ng/kg/min and 0.5 mg/kg/min, preferably between 10 ng/kg/min and 100 ng/kg/min.

The inventive composition may be administered for a period of at least one month, preferably at least two months, more preferred at least three months.

According to a specific aspect, the patient undergoing therapy is suffering from mild VOD.

According to a specific aspect, the patient undergoing therapy is suffering from moderate VOD. Specifically, the composition is administered to a patient who is at risk of VOD, preferably following therapy with chemotherapeutic agents, irradiation, anti-CD33 targeting immunotoxins, after long-term immunosuppression with azathioprine in kidney or liver transplantation or haematopoetic stem cell (HES) transplantation.

Chemotherapeutic agents that are known to induce VOD are for example, but not limited to, vincristine, dactinomycin (actinomyocin D), doxorubincin, cyclophosphamide, etoposide, dacarbazine, cytosine arabinoside, mithramycin (plicamycin), 6-thioguanine, urethane and indicine N-oxide, alone. Milder forms of liver disease from chemotherapy which share the key aspect of sinusoidal endothelial cell injury include nodular regenerative hyperplasia, sinusoidal dilatation and peliosis hepatis.

Chemotherapeutic agents like vincristine, dactinomycin (actinomyocin D), doxorubincin and cyclophosphamide are specifically used for the treatment of Wilm's tumor and other childhood kidney tumors. For example, treating nephroblastoma (Wilms' tumor) with dactinomycin and abdominal irradiation has led to VOD.

Combinations of irradiation and chemotherapy have also led to the development of VOD.

Radiation-induced liver disease is a condition that shares some of the features of VOD, although there are differences in clinical presentation, histology and time course.

Radiation-induced liver disease is seen with radiation doses in excess of to 35 Gy in adults.

More specifically, the patient suffering from hepatic VOD was primarily suffering from bone marrow disease, preferably he had undergone a HES transplantation.

The patient is herein specifically understood as a human being.

Specifically, the patient is suffering from any of the hepatic VOD or sinusoidal obstruction syndrome.

Further, according to the present invention there is provided a kit for treating or preventing hepatic VOD and/or sinusoidal obstruction syndrome in a patient, comprising
(i) an effective amount of a prostacyclin or prostacyclin analogue or derivative or a pharmaceutically acceptable salt thereof, specifically a pharmaceutically acceptable salt of Treprostinil;
(ii) one or more pharmaceutically acceptable carriers and/or additives; and
(iii) instructions for use in treating VOD.

The present invention also provides a method for preventing or treating the sinusoidal obstruction syndrome and/or hepatic veno-occlusive disease (VOD) by administering a composition comprising a prostacyclin analogue or derivative or a pharmaceutically acceptable salt thereof to a subject.

### FIGURES

FIG. 1

Quantification of mRNAs encoding prostaglandin receptors by liver cells.

FIG. 2

Concentration-response curve for busulfan-induced death of TSECs.

FIG. 3

Attenuation by treprostinil of busulfan-induced cell death in TSECs.

FIG. 4

Attenuation of busulfan-induced cell death in TSECs—titration of treprostinil.

FIG. 5

Effect of incubation time and busulfan concentration on the yield of mRNA from TSECs

FIG. 6

Stimulation of cAMP accumulation in TSECs: cAMP accumulation was assessed in 1×10⁶TSECs, which were plated in duplicates and treated with treprostinil or the combination thereof with forskolin at the indicated concentrations for 1 h. Control cells were incubated in standard medium.

### DETAILED DESCRIPTION OF THE INVENTION

It has been surprisingly found by the inventors that prostacyclin or prostacyclin analogues or derivatives or a pharmaceutically acceptable salt thereof can be used for treating hepatic VOD and/or sinusoidal obstruction syndrome.

Sinusoidal obstruction syndrome is a new name given to VOD of the liver, since VOD can develop without venular involvement.

The pathophysiology of VOD is very complex and multiple factors are involved. From a cell biological perspective the complex nature of the disease implicates the involvement of multiple signalling pathways. Thus ideally, the treatment concept should simultaneously target several nods in the signal network central to the development of VOD.

Intracellular cAMP is known to impinge on the regulation of many genes including those required to mount an inflammatory response and to fend off activated clotting factors and activated platelets. In addition, cAMP-dependent phosphorylation regulates the activity of apoptotic pathways (e.g., by inactivating the proapoptotic protein BAD). Thus, cAMP is an interesting downstream target in the treatment of VOD, because it allows for addressing several responses that are relevant in VOD. There are many receptors, which are coupled to Gₛ and thus to cAMP elevation. Prostacyclin analogues are specifically advantageous because said compounds can prevent the severe complication VOD and can also improve the outcome of haematopoietic stem cell transplantation. The safety profile of Treprostinil is well known; this further justifies its application in a preventive setting. Last but not least, Treprostinil is a stable and defined compound whereas other compounds like Flolan are instable, have a very short serum half-life and have therefore a short efficacy period. Additionally, Flolan stimulates prostaglandin IP receptors, which are of very low expression in sinusoidal endothelial cells.

Synthetic prostacyclin analogues can be, for example, but are not limited to, Treprostinil, Iloprost, Cicaprost or Beraprost, preferably Treprostinil is used.

The synthetic prostacyclin I2 (PGI₂) analogues like for example Treprostinil, Iloprost, Beraprost and Cicaprost are capable of increasing cAMP levels in cells. Specifically, Treprostinil is a stable analogue of prostacyclin/PGI₂, which also selectively stimulates EP₂- and EP₄-receptors but has only low affinity to EP₃ receptors. Thus it has the potential to stimulate multiple Gₛ-coupled receptors while not engaging inhibitory (i.e., Gᵢ-coupled) EP₃-receptors, which inhibit cAMP accumulation. For the latter, dimethyl-PGE₂ is a full agonist. In contrast, Treprostinil is only a low affinity agonist at EP₃-receptors.

Because they are metabolically more stable than natural prostacyclins, Treprostinil, Iloprost, Beraprost and Cicaprost can elicit long lasting effects and prolonged/repeated administration of a prostacyclin analogue, specifically of Treprostinil, Iloprost, Beraprost and Cicaprost are well tolerated.

Suitable prostacyclin derivatives include but are not limited to acid derivatives, pro-drugs, sustained release forms, inhaled forms and oral forms of Treprostinil, Iloprost, Cicaprost or Beraprost.

A pharmaceutically acceptable salt of a prostacyclin or prostacyclin analogue of this invention can be formed between an acid and a basic group of the compound, such as an amino functional group, or a base and an acidic group of the compound, such as a carboxyl functional group. According to another embodiment, the compound is a pharmaceutically acceptable acid addition salt.

Specifically, Treprostinil or its derivative is useful according to the invention. Treprostinil can successfully enhance chloride channel function in epithelial cells of the lung of cystic fibrosis patients. Specifically the active agent, e.g. Treprostinil, is indicated for long-term therapy in mild to moderate VOD cases.

Treprostinil is a synthetic analogue of prostacyclin. Treprostinil is marketed as Remodulin™. Treprostinil is a (1R,2R,3aS,9aS)-[[2,3,3a,4,9,9a-hexahydro-2-hydroxy-1-[(3S)-3-hydroxyoctyl]-1H-benz[f]inden-5-yl]oxy]acetic acid monosodium salt. Iloprost is marketed as “Ilomedine” and is a 5-{(E)-(1S,5S,6R,7R)-7-hydroxy-6[(E)-(3S,4R5)-3-hydroxy-4-methyl-1-octen-6-inyl]-bi-cyclo[3.3.0]octan-3-ylidene}pentanoic acid.

Beraprost is a 2,3,3a,8b-tetrahydro-2-hydroxy-1-(3-hydroxy-4-methyl-1-octen-6-ynyl)-1H-cyclopenta(b)benzofuran-5-butanoic acid.

Cicaprost is a 2-[(2E)-2-[(3aS,4S,5R,6aS)-5-hydroxy-4-[(35,45)-3-hydroxy-4-methylnona-1,6-diynyl]-3,3a,4,5,6,6a-hexahydro-1H-pentalen-2-ylidene]ethoxy]acetic acid.

According to a specific embodiment, at least two, specifically at least three, four, five or six, or even more different prostacyclin analogues can be used. The composition of the invention can also comprise Treprostinil together with one or more of Iloprost, Cicaprost or Beraprost. Alternatively the composition can comprise Iloprost in combination with one or more of Treprostinil, Cicaprost or Beraprost or pharmaceutically acceptable salts thereof. Alternatively, the composition can comprise Beraprost in combination with one or more of Treprostinil, Cicaprost or Iloprost or pharmaceutically acceptable salts thereof. Alternatively, the composition can comprise Cicaprost in combination with one or more of Treprostinil, Beraprost or Iloprost or pharmaceutically acceptable salts thereof.

In reference to prostacyclin analogues, according to the present invention the term “prostacyclin analogues” includes derivatives and analogues of said substances.

The terms “analogue” or “derivative” relate to a chemical molecule that is similar to another chemical substance in structure and function, often differing structurally by a single element or group, which may differ by modification of more than one group (e.g., 2, 3, or 4 groups) if it retains the same function as the parental chemical. Such modifications are routine to skilled persons, and include, for example, additional or substituted chemical moieties, such as esters or amides of an acid, protecting groups such as a benzyl group for an alcohol or thiol, and tert-butoxylcarbonyl groups for an amine. Also included are modifications to alkyl side chains, such as alkyl substitutions (e.g., methyl, dimethyl, ethyl, etc.), modifications to the level of saturation or unsaturation of side chains, and the addition of modified groups such as substituted phenyl and phenoxy. Derivatives can also include conjugates, such as biotin or avidin moieties, enzymes such as horseradish peroxidase and the like, and radio-labeled, bioluminescent, chemoluminescent, or fluorescent moieties. Further, moieties can be added to the agents described herein to alter their pharmacokinetic properties, such as to increase half-life in vivo or ex vivo, or to increase their cell penetration properties, among other desirable properties. Also included are prodrugs, which are known to enhance numerous desirable qualities of pharmaceuticals (e.g., solubility, bioavailability, manufacturing, etc.).

The term “derivative” also includes within its scope alterations that have been made to a parent sequence including additions, deletions, and/or substitutions that provide for functionally equivalent or functionally improved molecules.

According to the invention, the term “about” includes a deviation of the numerical value of a maximum of 10%, specifically a maximum of 5%, more specifically a maximum of 1%.

According to a specific embodiment of the invention, the Treprostinil derivative is selected from the group of acid derivatives of Treprostinil, prodrugs of Treprostinil, polymorphs of Treprostinil or isomers of Treprostinil.

Similarly, Iloprost, Cicaprost or Beraprost can be derivatives from the group of acid derivatives, prodrugs, polymorphs or isomers therefrom.

Specifically, physiologically acceptable salts of Treprostinil include salts derived from bases. Base salts include ammonium salts (such as quaternary ammonium salts), alkali metal salts such as those of sodium and potassium, alkaline earth metal salts such as those of calcium and magnesium, salts with organic bases such as dicyclohexylamine and N-methyl-D-glucamine, and salts with amino acids such as arginine and lysine.

Treprostinil is a stable analogue of prostacyclin (PGI₂), and in addition to prostanoid I receptor it stimulates EP₂- and EP₄-receptors (Whittle et al., 2012). Hence, any direct therapeutic impact in VOD can rely on the expression and engagement of Gₛ-coupled receptors in target cells of the diseased liver.

Treprostinil is of high metabolic stability which specifically allows for administration by various routes.

According to the invention the derivatives of Treprostinil can be, for example, acid derivatives of Treprostinil, prodrugs of Treprostinil, sustained release forms of Treprostinil, inhaled forms of Treprostinil, oral forms of Treprostinil, polymorphs of Treprostinil or isomers of Treprostinil.

The composition of the invention can be present in any form which can be used for administration, in particular as pharmaceutical preparation.

For example, the composition of the invention can be administered as liquid or powder. It can be administered topically, intravenously, subcutaneously, by inhalation, e.g. to administer an aerosol, or by using a nebulizer, or in orally available form like tablets or capsules. Due to the high metabolic stability of some prostacyclin analogues like Treprostinil, or if provided as lipid based or pegylated forms of the prostacyclin or prostacyclin analogues, the substances can also be administered as depot medicaments.

Aerosolized delivery of the prostacyclin analogue may result in a more homogeneous distribution of the agent in a lung, so that deep lung delivery is obtained. Thereby the dosage of application might be reduced to the sustained presence of the agent at the site of action in the lung.

The composition can be administered with any pharmaceutically acceptable substances or carriers or excipients as known in the art. These can be for example, but are not restricted to water, neutralizing agents like NaOH, KOH, stabilizers, DMSO, saline, betaine, taurine etc.

The term “pharmaceutically acceptable” means approved by a regulatory agency of the Federal or a state government or listed in the U.S.

The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatine, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. The formulation should be selected according to the mode of administration.

The amount of the active drug in the inventive composition can be selected by any skilled person, preferably an amount of the prostacyclins or prostacyclin analogues or pharmaceutically acceptable salts thereof, specifically of Treprostinil, which is in the range of 0.1 ng/kg to 0.5 mg/kg of body weight/min, specifically it may be >0.5 mg/kg of body weight/min.

Treprostinil or any other prostacyclin analogue according to the invention is to be administered in an effective amount. Said effective amount can be determined by the skilled person, i.e. by weighing the therapeutic effect on the VOD symptoms and possible side effects that may occur by overdosing the compound.

According to an embodiment the patient is treated with Treprostinil at a daily dose of 0.1 to 10 mg preferably by continuous subcutaneous infusion.

In an embodiment, the prostacyclin analogue, specifically Treprostinil, is administered to a patient at risk of or suffering from hepatic VOD at a dose to improve the patient's liver function and/or normalize coagulation time.

According to a further embodiment, the composition is administered to a patient suffering from mild to moderate hepatic VOD.

According to an embodiment of the invention, the patient having mild VOD is characterized by up to 5%, specifically up to 10% weight increase and/or a maximum total serum bilirubin of up to 7 mg/dl before day 20 and/or having a risk of developing peripheral edema of up to 25%, specifically up to 50% and/or up to 50% platelet transfusion requirement to day 20.

According to an embodiment of the invention, the patient having moderate VOD is characterized by up to 10%, specifically up to 15% weight increase and/or a maximum total serum bilirubin of up to 7%, specifically up to 10 mg/dl, specifically up to 20%, specifically up to 25 mg/dl before day 20 and/or having a risk of developing peripheral edema of up to 25%, specifically up to 50% and/or up to 80% specifically up to 90%, specifically up to 100% platelet transfusion requirement to day 20.

One of the targets for intervention in VOD may be thrombosis events. The prostacyclin, prostacyclin analogue, derivative or pharmaceutically acceptable salt thereof according to the invention, specifically Treprostinil, can provide an advantageous effect in the treatment of hepatic VOD compared to prostaglandin E₁ (PGE₁) which was already tested in clinical studies for the treatment of VOD, also in combination with heparin, but could not demonstrate any beneficial effect and administration of PGE₁ was complicated by significant toxicity.

As used herein, the patient to be treated can be any mammal, but preferably the mammal is a human, a non-human primate, a rodent, a cow, a horse, a sheep, or a pig. Other mammals can also be treated in accordance with the present invention.

The invention further provides a kit for treating or preventing a condition associated with hepatic VOD or sinusoidal obstruction syndrome in a subject, comprising (i) an effective amount of a prostacyclin or prostacyclin analogue or derivative or a pharmaceutically acceptable salt thereof, (ii) one or more pharmaceutically acceptable carriers and/or additives, and (iii) instructions for use in treating or preventing VOD.

According to a specific embodiment of the invention, the kit comprising (i) an effective amount of a prostacyclin or prostacyclin analogue or a pharmaceutically acceptable salt thereof, (ii) one or more pharmaceutically acceptable carriers and/or additives, and (iii) instructions for use in treating or preventing hepatic OD or sinusoidal obstruction syndrome is provided for use in the treatment or prevention of a VOD or sinusoidal obstruction syndrome in a patient.

Said component (i) can be in a form suitable for intravenous administration, for inhalation, or for oral administration.

More specifically, the present invention provides the use of a kit wherein the active agent or ingredient is Treprostinil or a pharmaceutically acceptable salt thereof. Specifically, the component (i) comprises a pharmaceutically acceptable salt of

Treprostinil.

Clinical tests may be performed to determine the suitable dose/treatment regimen in human beings.

It is envisaged that treatment with the composition according to the invention may follow therapy with chemotherapeutic agents, irradiation, anti-CD33 targeting immunotoxins, or haematopoetic stem cell (HES) transplantation.

Additionally, the patient may be suffering from bone marrow disease, preferably a patient who was undergoing HES transplantation and developed the disease after the transplantation.

In an embodiment of the invention, a patient is continuously treated with the composition for a period of at least several days, preferably at least 1 month, preferably at least 2 months, more preferred at least 3 months.

The examples described herein are illustrative of the present invention and are not intended to be limitations thereon. Different embodiments of the present invention have been described according to the present invention. Many modifications and variations may be made to the techniques described and illustrated herein without departing from the spirit and scope of the invention. Accordingly, it should be understood that the examples are illustrative only and are not limiting upon the scope of the invention.

### EXAMPLES

### Example 1

Expression of mRNAs Encoding Prostaglandin Receptors by Liver Cells.

Hepatic sinusoidal endothelial cells (HSECs) are a unique subpopulation of fenestrated endothelial cells lining the hepatic sinusoids and comprise the majority of endothelial cells within the liver. It is time-consuming to isolate primary sinusoidal endothelial cells from mouse liver, they are limited in number and difficult to maintain in a differentiated state under cell culture conditions. Huebert R C et al (Lab Invest 90(12):1770-81, 2010) generated an immortalized cell line derived from hepatic sinusoidal endothelial cells (HSECs), which retains an endothelial phenotype but also mimics pathological vasculature. This cell line of transformed sinuendothelial cells (TSECs) was generated by immortalization with SV40 large T-antigen. TSECs are suitable to study VOD: 1) in spite of their immortalized nature, they retain a number of key endothelial features, including migration in response to angiogenic factors, formation of vascular tubes, endocytosis and remodelling of extracellular matrix, 2) their phenotype resembles several pathological features associated with chronic liver disease but also with VOD, in which cells become activated, proliferative, defenestrated and angiogenic. Thus, some of the morphological features of TSECs are also evident in livers of VOD patients.

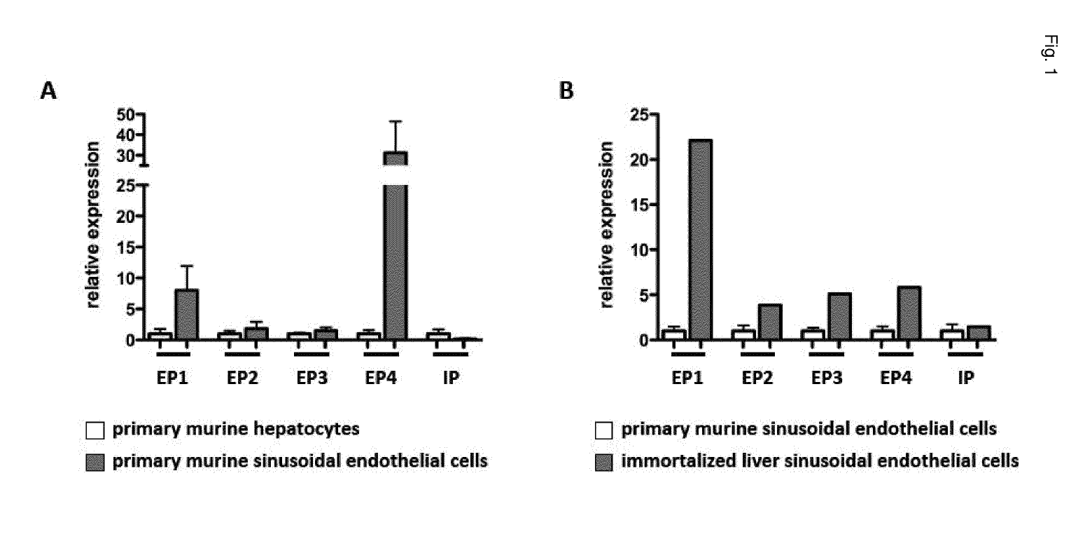

Treprostinil is a stable analogue of prostacyclin (PGI₂), and in addition to prostanoid I receptor it stimulates EP₂- and EP₄-receptors (Whittle et aL, 2012). Hence, any direct therapeutic impact in VOD would rely on the expression and engagement of Gₛ-coupled receptors in target cells of the diseased liver. Therefore we first verified target receptor expression in our model cell line. RNA was isolated from the immortalized liver sinusoidal endothelial cell line (TSEC, FIG. 1 panel B). We included primary murine hepatocytes and sinusoidal endothelial cells in this experiments (FIG. 1, panel A). Complementary DNA (cDNA) was generated and levels of transcripts encoding prostaglandin receptors (EP₁, EP₂, EP₃, EP₄ and IP) were assessed by qPCR (quantitative polymerase chain reaction). As illustrated in FIG. 1, amplicons of all receptors were detected in both, the TSEC model cell line as well as murine primary sinusoidal endothelial cells. Thus, TSECs can be used to study the effects of treprostinil. In addition, because EP₁ and EP₄ receptors are expressed in primary murine sinusoidal endothelial cells, it should be possible to carry out bridging experiments, in which experimental findings observed in human TSECs are recapitulated in primary murine cells in vitro and linked to murine in vivo models.

RNA was isolated from murine primary liver cells, i.e. hepatocytes and sinusoidal endothelial cells (LSECs; panel A) and from an immortalized liver sinusoidal endothelial cell line (TSEC) with stable expression of SV40 large T-antigen (panel B) using TRIO Reagent RNA Isolation Reagent (Sigma Aldrich, Austria) or the NucleoSpin RNA II kit from Machery-Nagel following the instructions of the manufacturer. Complementary DNA (cDNA) was generated using the High Capacity cDNA Reverse Transcription Kit (Applied Biosystems) following the instructions of the manufacturer. The levels of transcripts encoding prostaglandin receptors (EP1, EP2, EP3, EP4 and IP) were assessed by qPCR (quantitative polymerase chain reaction) using 5 μL of cDNA in a final reaction volume of 20 μL. Amplicons of all receptors were detected in all analyzed cell types. For the sake of comparison in panel A, the levels of each transcript in the primary murine hepatocyte were set 1 and the level in sinusoidal cells normalized to these levels. This comparison highlights that the mRNA levels of EP4, a target receptor for Treprostinil, were expressed at higher levels in LSECs compared to hepatocytes (panel A). Panel B compares mRNA expression of different prostaglandin receptors between primary LSECs and TSEC with stable expression of SV40 large T-antigen by using levels in LSECs as the reference. The levels of mRNA encoding prostaglandin receptor EP1-4 were higher in TSECs compared to LSECs. This observation confirms that TSECs are a suitable model system to study the potential protective and/or curative effects of treprostinil in VOD.

The data are shown in FIG. 1.

### Example 2

Concentration-Response Curve for Busulfan-Induced Death of TSECs.

In clinical practice, conditioning regimes based on high-dosed busulfan are associated with an increased risk of VOD, which is related to the toxicity of busulfan

(Cheuk D K et al, Bone Marrow transplant 40: 935-944, 2007). We tested, if the toxicity of busulfan was amenable to in vitro studies on TSECs. Accordingly, we defined the concentration-response curve for busulfan-induced death of TSECs. In analogy to Raimer J et al (EurJClinPharmacol 68: 932-935, 2012), who had used ECV304 cells, we plated TSECs on collagen-coated plastic dishes (5×10³ cells/24-well plate). After 24 hours, busulfan was added at increasing concentrations (10 μM-1 mM). The concentration range was based on reports in the literature and on the serum levels observed in patients undergoing myeloablative chemotherapy. Cell viability was assessed after 24, 48 and 72 h. No obvious cell death was observed after 24 h. After 72 h, almost complete cell death was also observed at lower concentrations.

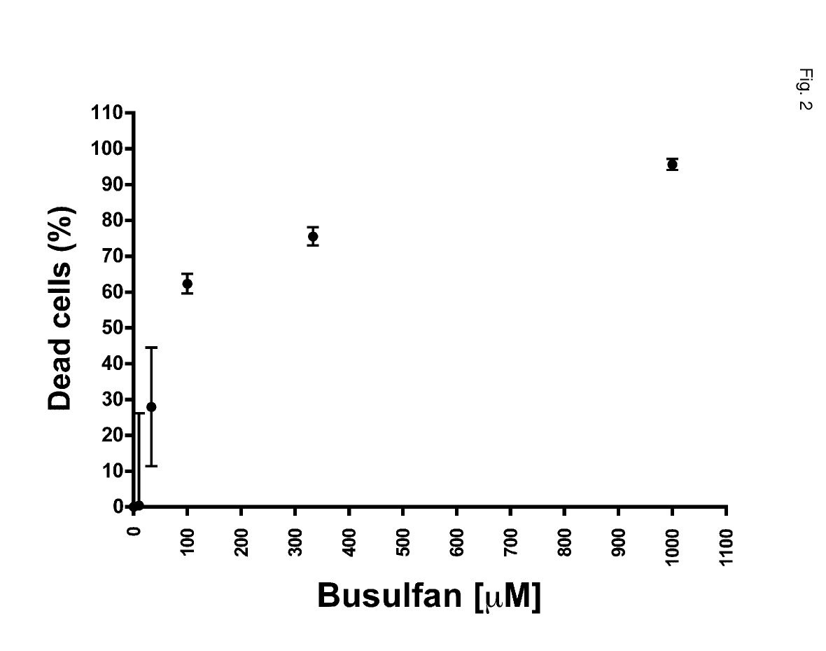

At the very least this experiment addresses the question, if busulfan can per se induce damage in TESCs or if the presence of hepatocytes, e.g. in a co-culture, is required to produce the toxic intermediate(s). If the latter had been the case, a glutathione conjugate of busulfan and the metabolite 1,4-diiodobutane would have been available (Marchand D H et al, Drug Metab Dispos 16(1):85-92, 1988). However, as can be seen from FIG. 2, busulfan per se induced cell death in a concentration dependent manner in TESCs. Thus, this observation suggests that the effects of busulfan can be studied in the absence of hepatocytes. This reduces the complexity of the in vitro model.

TSECs were plated on collagen-coated plastic dishes (5*10³ cells/24-well plate). After 24 hours busulfan, a cell cycle non-specific alkylating antineoplastic agent, in the class of alkyl sulfonates with a chemical designation is 1,4-butanediol dimethanesulfonate, used as chemotherapeutic agent and known to induce VOD, was added at increasing concentrations (10 μM-1 mM). The concentration range is based on reports in the literature and on the serum levels observed in patients undergoing myeloablative chemotherapy. Cell viability was assessed after 24, 48 and 72 hours. For this purpose cells were washed twice with PBS, trypsinized, mixed with trypan blue (1:1 volumes) and counted using a hemocytometer. No obvious cell death was observed after 24 hours. The data (means±standard deviation) shown represent cell death after for 48 hours. After 72 hours, almost complete cell death was also observed at lower concentrations.

The data are shown in FIG. 2.

### Example 3

Attenuation of Busulfan-Induced Cell Death in TSECs with Treprostinil.

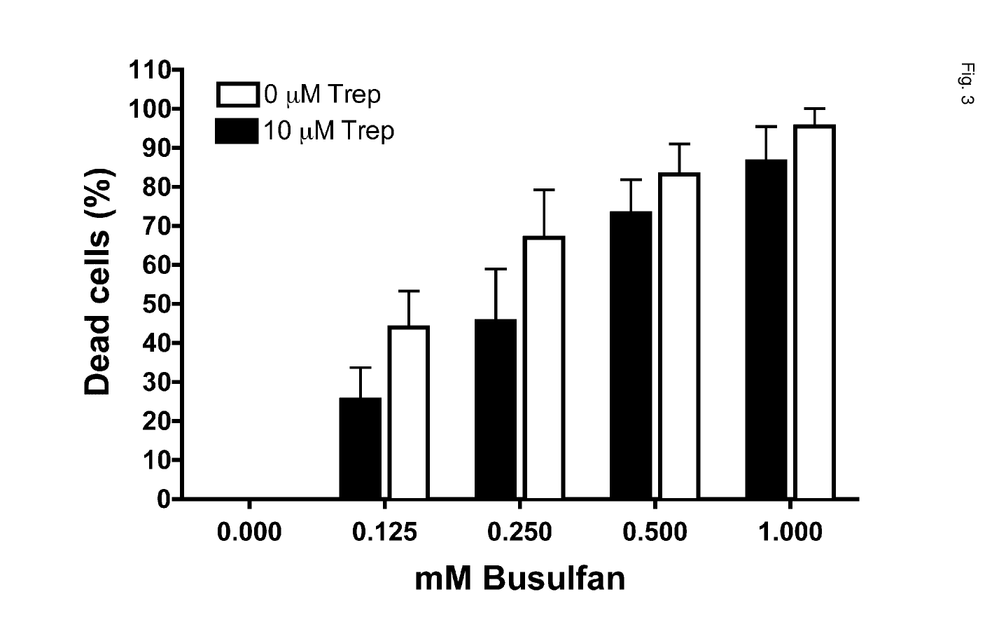

TSECs were pretreated with treprostinil (10 μM) for 1 h and examined, if this protected them against busulfan-induced cell death by challenging them—in the continuous presence of treprostinil—with 125 and 250 μM and 500 μM Busulfan (BU). After 24 h, cell viability was determined by counting detached cells in a haemocytometer and determining the number of trypan blue-positive (=dead) and trypan-blue excluding (=live) cells. As documented in FIG. 3, 10 μM treprostinil attenuated BU-induced toxicity. This effect was observed in three independent experiments.

TSECs were plated on collagen-coated plastic dishes (5*10³ cells/24-well plate). After 24 hours treprostinil was added at a concentration of 10 pM. One hour later, busulfan was added at increasing concentrations. Cell viability was assessed after 48 hours. Treatment with treprostinil was associated with increased viability, if the cells were exposed for 48 hours to busulfan at concentrations of 125 and 250 μM.

The data are shown in FIG. 3.

### Example 4

Attenuation of Busulfan-Induced Cell Death in TSECs—Titration of Treprostinil

We examined the concentration range, in which treprostinil protected TSECs against busulfan-induced cell death. TSECs were plated on collagen-coated plastic dishes and after 24 h treprostinil was added at concentrations of 5, 10 and 20 pM. One hour later, busulfan was added at concentrations of 125 and 250 μM. Cell viability was assessed after 48 h. Even low concentrations of treprostinil (5 μM) attenuated busulfan-induced cell death. Both, busulfan (p<0.001) and treprostinil (p=0.0112) had a significant effect on cell viability. The data were analyzed using a 2-way ANOVA. The concentration range, which was examined did not suffice to define a concentration-response relation because the methods are not sensitive enough for a binding statement on the concentration response relation, however, the observations suffice to document that at a concentration of 10 μM treprostinil is close to saturation.

TSECs were plated on collagen-coated plastic dishes (5*10³ cells/24-well plate). After 24 hours treprostinil was added at concentrations of 5, 10 and 20 pM. One hour later, busulfan was added at concentration of 125 and 250 μM. Cell viability was assessed after 48 hours. Even low concentrations of treprostinil (5 μM) attenuated busulfan induced cell death. This beneficial effect did not increase at higher concentrations due to the sensitivity of the measurement methods. The data were analyzed by two-way ANOVA. A Bonferroni post hoc test was conducted to test for differences between individual treatment groups. Both, busulfan (p<0.001) and treprostinil (p=0.0112) had a significant effect on cell viability.

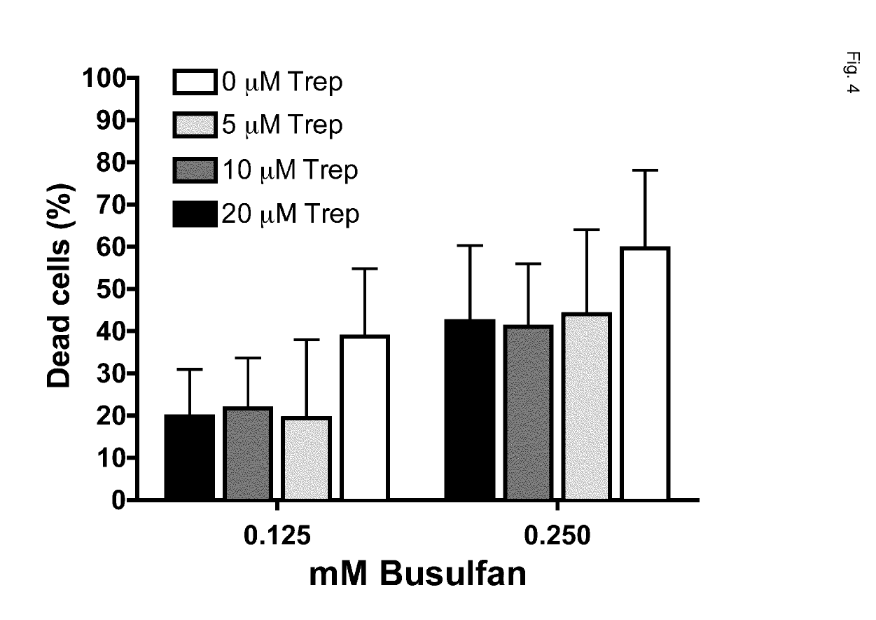

The data are shown in FIG. 4.

Experiments designed to examine busulfan- and Treprostinil-mediated effects on target genes involved in blood coagulation:

### Example 5

Effects of Busulfan on Transcripts of Genes Associated with Coagulation

In EC340 cells, busulfan alters the expression of several genes, which are known to be deregulated in VOD. Cyclic AMP is involved in the regulation of some of these genes, in particular in the regulation of PAI-1 (e.g. DiBattista J A et al, Mol Cell Endocrinol 103(1-2):139-48, 1994, Sunagawa M et. al., Endothelium 13 (5): 325-33, 2006). Hence, we aimed at testing whether treprostinil counteracts some actions of busulfan on gene expression. PAI-1 expression however is also regulated by growth factors and cytokines (e.g. Heaton J H et al, JBC 5; 273 (23):14261-8, 1998, summarized in Heaton J H, Dlakic W M, Gelehrter T D Thromb Haemost 89(6):959-66, 2003).

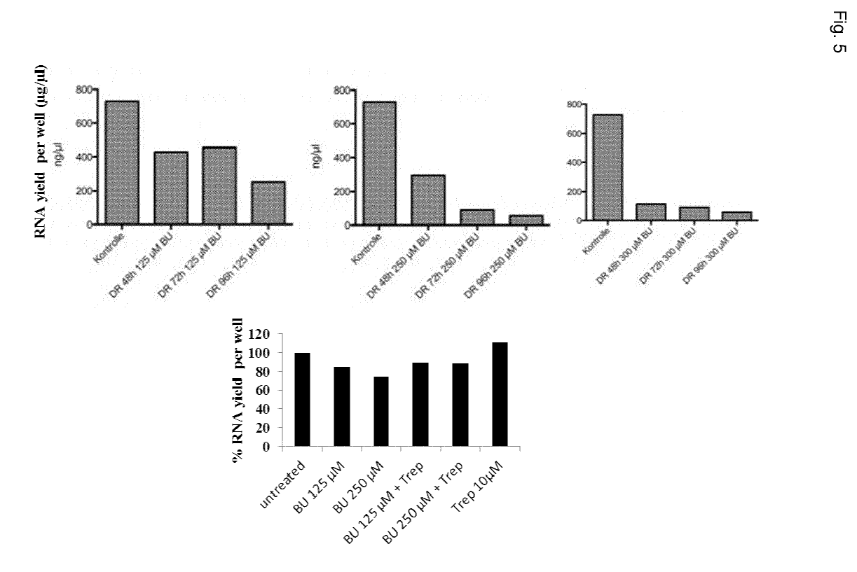

We first defined the concentration range of busulfan, which triggered a change in expression profile but did not yet kill the cells. In EC340 cells, concentrations as high as 300 μM were tolerated for 96h and allowed for investigating target gene expression and regulation upon treatment with busulfan. This concentration was not compatible with the extraction of mRNA in TSECs: the amount, quality and stability of mRNA were affected in a dose- and time-dependent manner (FIG. 5 and data not shown). Pretreatment of TSECS with busulfan at 125 μM for 48 h was the highest time*concentration product, which allowed to harvest mRNA in a quality adequate for reliably and reproducibly assessing gene regulation by qPCR (FIG. 5). Based on the time course of gene regulation in EC340 cells, we tested a treatment duration between of a minimum of 12h and a maximum of 96h (data not shown).

FIG. 5 shows the effect of incubation time and busulfan concentration on the yield of mRNA from TSECs: 1.6×10⁵ TESCS were seeded per well. Cells were either treated with 125 μM, 250 μM or 300 μM BU, the combination thereof with treprostinil (10 μM), or treprostinil (10 μM) alone, or kept in standard medium [Endothelial Cell Medium, ECM Sciencell™, USA, TSECs containing 5% fetal bovine serum, 1% endothelial cell growth supplement (ECGS) and 1% penicillin/streptomycin at 37° C. and 5% CO₂]. a) Total RNA was extracted by Trizol® (lifetechnologies) according to the manufacturer's protocol. RNA concentration (ng/μl) and purity was determined with Nanodrop 2000®. If equal numbers of cells (1.6×10⁵) were seeded per well, treatment with 250 and 300 μM BU induced cell death in as much rendering reproducible and valid yields of absolute RNA content impossible, already after 48h of treatment. b) The absolute yield in RNA preparations (μg/μl) was compared between cells, which had been incubated in standard medium (untreated), and cells, which had been treated with BU (125 or 250 μM BU) plus treprostinil (10 μM Trep), or treprostinil alone.

FIG. 5a exemplifies results from these preliminary experiments. Equal numbers of cells (1.6×10⁵) were seeded per well. As expected, treatment with busulfan was toxic to the cells. More importantly, it became evident that a treatment with busulfan at concentrations of 300 μM and 250 μM precluded the isolation of RNA at acceptable yields and of acceptable quality after 48h of treatment. Even 125 μM of BU diminished the yield of RNA preparations per well, however the subsequent generation of cDNA was feasible in a reproducible manner.

Thus, for further experiments the following treatment regime was chosen: pretreatment of cells with 10 μM treprostinil for 1 h, followed by co-incubation of cells with 10 μM treprostinil and 125 μM BU for 24 h.

We noticed that treatment with treprostinil prevented the busulfan-induced reduction in the amount of RNA: as illustrated in FIG. 5b, treatment with 125 μM and 250 μM busulfan for 48h reduced the yield of RNA to 85 and 74%, respectively, as compared to untreated cells (100% RNA yield). However, if pretreated (1 hour) and co-incubated (24 h) with 10 μM treprostinil, this loss was diminished to at least some extent: the loss in RNA yield was lowered to 11%. Interestingly, if cells were treated with treprostinil alone, RNA content was increased to 111%, as compared to control cells. This finding was reproduced in 5 independent RNA preparations.

### Example 6

Identification of Suitable Candidate Target Genes in TSECs Subject to Regulation by Busulfan and Treprostinil

The expression of candidate target genes was examined to monitor the detrimental effects of busulfan and the protective effect of treprostinil.

After pretreatment of TESCs with 10 μM treprostinil for 1 h, cells were co-incubated with 10 μM treprostinil and 125 μM BU for 24 h. Total RNA was extracted by the use of PARIS Kit (life technologies). cDNA synthesis was performed by High-Capacity Reverse Transcription Kit (applied biosystems); the resulting cDNA (equivalent to 50 ng RNA/reaction) was analyzed via qPCR (StepOnePlus real-time PCR System®, Applied Biosystems). 18s rRNA was used as a reference gene.

Based on preliminary experiments we identified plasminogen-activator inhibitor-1 (PAI-1), tissue factor (TF), tissue factor pathway inhibitor (TFPI) and thrombomodulin (TM) as suitable genes to study the potential of treprostinil to counteract busulfan-induced effects in TSECs. We also tested if busulfan-mediated effects are counteracted by 10 μM treprostinil. A total of 1.5×10⁵ TESCs were plated in 6 well plates. As soon as 90% confluence was reached, cells were pretreated with treprostinil for 1 h, then cells were incubated for 24h in standard culture medium containing both BU and treprostinil. Thereafter, cells were washed and mRNA was prepared. Treprostinil counteracted the busulfan-induced increased expression of procoagulatory factors such as PAI-1 and TF (data not shown). These experiments document that busulfan induces the expression of factors, which favour blood coagulation—i.e., PAI-1 and TF—and are thus permissive for the development of VOD. Similarly, busulfan decreased the expression of the anticoagulant factor thrombomodulin (TM) and treprostinil mitigated the effect of busulfan (data not shown). These observations justify the use of TSECs as an in vitro model system to study surrogate parameters, which are predictive of an increased risk of VOD in vivo.

Interestingly, among the candidate genes, which were investigated, expression of TFPI was found to be induced by both, busulfan and treprostinil.

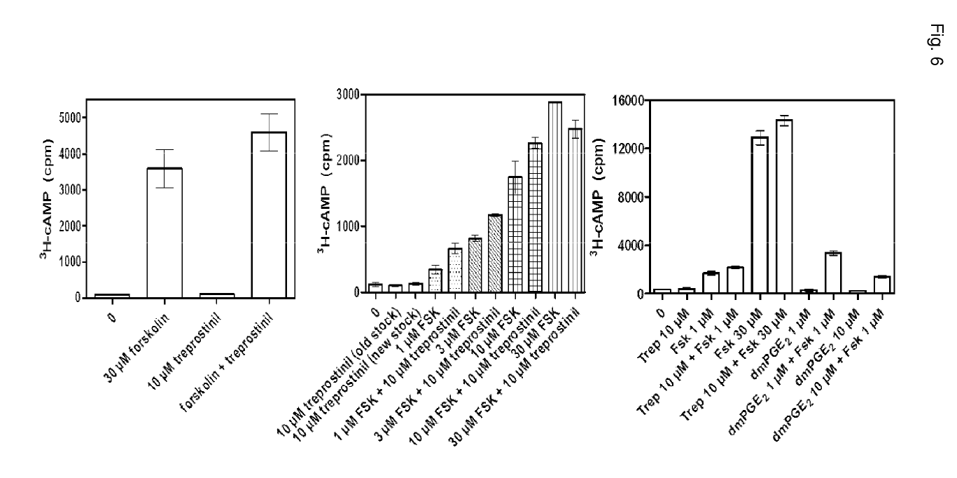

Treatment of TESCs was performed as indicated in the legend to FIG. 6. Treprostinil-treatment was initiated 1 h prior to addition of busulfan. Cells were collected 24 h later. The expression levels of mRNA were quantified by PCR and normalized to 18s RNA levels. a) Treprostinil counteracted the busulfane-induced increase in the expression of procoagulatory factors PAI-1 and TF and the busulfan-induced decrease in profibrinolytic TM. b) In TESCs, treprostinil regulates the expression of mRNA of TFPI in a concentration-dependent manner.

Thus, TFPI was selected to investigate the concentration-response curve of treprostinil. This experiment verified that the concentration range of 10 to 20 μM was most effective (data not shown).

### Example 7

Induction of cAMP Levels in TESCs with Treprostinil

It is desirable to understand the mechanisms, which allow treprostinil to modulate busulfan-induced gene expression, because this allows for searching for additional target genes. It was assumed that the action of treprostinil is mediated by an increased accumulation of cAMP. However, surprisingly, sole addition of treprostinil did not suffice to increase intracellular cAMP levels in TSECs (FIG. 6). However, if cells were sensitized with forskolin, treprostinil (10 μM) further increased intracellular cAMP levels in TESCs. This was seen both, in the presence of 30 μM forskolin (FIG. 8, left hand panel) and if the concentration of forskolin was titrated between 1 and 30 μM (FIG. 6, middle and right hand panel). It was also seen regardless of whether [³H]cAMP was low (FIG. 6, middle panel) or high (FIG. 6, right hand panel).

FIG. 6 shows the stimulation of cAMP accumulation in TSECs: cAMP accumulation was assessed in 1×10⁶TSECs, which were plated in duplicates and treated with treprostinil or the combination thereof with forskolin at the indicated concentrations for 1 h. Control cells were incubated in standard medium. For comparison, cAMP accumulation was also assessed after incubation with dmPGE2. The responses were comparable in the first and the second experiment. In the third experiment, the response was higher, presumably due to more extensive labelling of the adenine nucleotide pool. However, in none of the experiments tested, treprostinil alone sufficed to increase intracellular cAMP levels over that seen in untreated control cells.

1. A method of preventing or treating sinusoidal obstruction syndrome and/or hepatic veno-occlusive disease (VOD), comprising the step of administering to a subject in need thereof a composition comprising a prostacyclin analogue or a derivative or pharmaceutically acceptable salt thereof.

2. The method of claim 1, wherein said prostacyclin analogue is selected from the group consisting of treprostinil, iloprost, cicaprost, beraprost, and derivatives or pharmaceutically acceptable salts thereof.

3. The method of claim 1, wherein said derivative is selected from the group consisting of acid derivatives of treprostinil, prodrugs of treprostinil, sustained release forms of treprostinil, inhaled forms of Treprostinil, oral forms of treprostinil, polymorphs of treprostinil, and isomers of treprostinil.

4. The method of claim 1, wherein the composition is administered systemically.

5. The method of claim 1, wherein the composition is administered locally administration.

6. The method of claim 1, wherein said composition is in an orally available form selected from the group consisting of tablets and capsules.

7. The method of claim 2, wherein the treprostinil or its derivative or a pharmaceutically acceptable salt thereof is administered in an effective amount ranging between 0.1 ng/kg/min and 100 ng/kg/min.

8. The method of claim 2, wherein treprostinil is administered to a patient at risk of or suffering from VOD at a dose to improve the patient's liver function and/or normalize coagulation time.

9. The method of claim 1, wherein the composition is administered to a patient suffering from mild to moderate VOD.

10. The method of claim 1, wherein the composition is administered to a patient at risk of VOD following therapy with chemotherapeutic agents, irradiation, anti-CD33 targeting immunotoxins, or haematopoetic stem cell (HES) transplantation.

11. The method of claim 10, wherein the patient is suffering from bone marrow disease.

12. The method of claim 1, wherein the subject is continuously treated with the composition for a period of between 1 month and 3 months.

13. The method of claim 12, wherein the subject is treated with treprostinil at a daily dose of 0.1 to 10 mg.

14. (canceled)

15. The method of claim 4, wherein the composition is administered by intravenous or subcutaneous infusion.

16. The method of claim 5, wherein the composition is administered by inhalation.

17. The method of claim 10, wherein the patient is undergoing HES transplantation.

18. The method of claim 12, wherein the subject is continuously treated with the composition for a period of at least 2 months.

19. The method of claim 13, wherein the subject is treated by continuous subcutaneous infusion.
